(19) United States
(12) Patent Application Publication
(10) Pub. No.: US 2022/0048995 A1
(43) Pub. Date: Feb. 17, 2022

(54) USE OF AN INHIBITOR OF NTSR1 ACTIVATION OR EXPRESSION FOR PREVENTING WEIGHT LOSS, MUSCLE LOSS, AND PROTEIN BLOOD LEVEL DECREASE IN SUBJECTS IN NEED THEREOF

(71) Applicants: INSERM (INSTITUT NATIONAL DE LA SANTÉ ET DE LA RECHERCHE MÉDICALE), Paris (FR); UNIVERSITÉ DE PARIS, Paris (FR)

(72) Inventors: Patricia FORGEZ, Paris (FR); Olle MELANDER, Malmö (SE)

(21) Appl. No.: 17/274,597

(22) PCT Filed: Sep. 9, 2019
(86) PCT No.: PCT/EP2019/073991

§ 371 Date: Mar. 9, 2021

(30) Foreign Application Priority Data
Sep. 10, 2018 (EP) 18306182.9

(51) Int. Cl. C07K 16/28; A61P 21/06

(52) U.S. Cl. CPC C07K 16/286; A61P 21/06; A61K 2039/505; C07K 2317/56; C07K 2317/565

(57) ABSTRACT

Cachexia is a potentially lethal syndrome afflicting mammals, frequently complicates the treatment of infection, inflammation and cancer. It is characterized by involuntary weight loss, including muscle loss and decrease in protein blood level content. The inventors now show in 2 animal models (mice fed with normal diet and mice fed with high fat diet) that neutralisation of the long fragment of neurotensin with an inhibitor of NTSR1 activation or expression prevents weight loss, muscle loss and protein blood level decrease. Accordingly, the present invention relates to use of an inhibitor of NTSR1 activation or expression for preventing weight loss, muscle loss, and protein blood level decrease in subjects in need thereof.

### FIELD OF THE INVENTION

The present invention relates to use of an inhibitor of NTSR1 activation or expression for preventing weight loss, muscle loss, and protein blood level decrease in subjects in need thereof.

### BACKGROUND OF THE INVENTION

Cachexia is a potentially lethal syndrome afflicting mammals, frequently complicates the treatment of infection, inflammation and cancer. Cachexia may result from diverse causes such as age, cancer, and infections by parasites and by microorganisms such as bacteria, fungi, viruses and protozoa. Both acute and chronic infections and illnesses frequently cause cachexia. In particular, cancer-induced cachexia is the immediate cause of death in about 15% of cancer patients (1, 2). It is characterized by involuntary weight loss that is resistant to nutritional supplementation. Weight loss starts with the breakdown of white adipose tissue (WAT) mediated by the lipolytic enzymes adipose triglyceride lipase (Atgl) and hormone-sensitive lipase (Hsl) as well as loss of skeletal muscle. WAT lipolysis is believed to be induced by tumour-derived factors, such as tumour necrosis factor alpha (TNF-α) and interleukin (IL-) 6. After an initial reduction of tumour mass, treatment with chemotherapeutic agents frequently exacerbates cachexia, hampering further treatment and increasing mortality. There is an urgent need for treatment regimens that counter the development of cachexia.

Neurotensin (NTS), a 13-amino acid peptide, predominantly localized in specialized enteroendocrine cells of the small intestine and released by fat ingestion (3-6). NTS was initially described as a hormone in the gastrointestinal tract. It participates in the digestion of food through many actions, such as stimulating pancreatic and biliary secretions, inhibiting motility of the small intestine and gastric secretions, and facilitating the translocation of fatty acid (7). The effects of NT are mediated through three known NT receptors (NTR1, 2 and 3; also known as NTSR1, 2, and NTSR3, respectively (8).

NTS is strongly involved in the regulation of the energy balance because it regulates food intake in the lateral areas of the hypothalamus (9). It is also a regulator of ingestive and locomotor behaviors. The injection of NTS into the peripheral circulation decreases food intake, and NTS circulating rate is increased after bariatric surgery (10, 11). Recently it was shown that circulating leptin, an adipokine which regulates appetite and energy balance, was associated with higher proNTS levels. Therefore independently of NTS effects on central leptin signaling, NTS influence energy balance by modulating circulating leptin concentration. (12). Overall, NTS is considered as an anorectic peptide.

The NTS also activates the corticotropic axis, at the central level it increases the plasma levels of ACTH and corticosterone (13). On the periphery, NTS has a direct action on the adrenals gland and increase in CRH, ACTH, aldosterone and corticosterone released. (14, 15)

These hormones are involved in the muscle mitochondria energetic metabolism, with a disruption of muscle mitochondria induced by glucocorticoids

A recent study showed that NT-deficient mice demonstrate significantly reduced intestinal fat absorption and are protected from obesity, hepatic steatosis and insulin resistance associated with high fat consumption (Li J. et al. Nature. 2016 May 19; 533 (7603):411-5). Remarkably, in humans, the same authors show that both obese and insulin-resistant subjects have elevated plasma concentrations of pro-NT, and in longitudinal studies among non-obese subjects, high levels of pro-NT denote a doubling of the risk of developing obesity later in life (Li J. et al. Nature. 2016 May 19; 533(7603):411-5). All these results point out the fact that inhibiting neurotensin would be suitable for the treatment of obesity.

### SUMMARY OF THE INVENTION

The present invention relates to use of an inhibitor of NTSR1 activation or expression for preventing weight loss, muscle loss, and protein blood level decrease in subjects in need thereof. In particular, the present invention is defined by the claims.

### DETAILED DESCRIPTION OF THE INVENTION

The inventors surprisingly showed that treatment with an inhibitor of NTSR1 activation or expression prevents in vivo weight loss, muscle loss and protein blood level decrease.

Accordingly, the first object of the present invention relates to a method of preventing weight loss, muscle loss and/or protein blood level decrease in a subject in need thereof comprising administering to the subject a therapeutically effective amount of an inhibitor of NTSR1 activation or expression.

In particular, the present invention relates to an inhibitor of NTSR1 activation or expression for use in a method of preventing weight loss, muscle loss and/or protein blood level decrease in a subject in need thereof.

Accordingly, in some embodiments, the subject is underweight. As herein, the term “underweight” refers to a subject having a body mass index of below 18.5. As used herein, the term “body mass index” has its general meaning in the art and refers to refers to the ratio which is calculated as body weight per height in meter squared (kg/m²). The BMI provides a simple means of assessing how much an individual's body weight departs from what is normal or desirable for a person of his or her height. Common definitions of BMI categories are as follows: starvation: BMI—less than 15 kg/m²; underweight—BMI less than 18.5 kg/m²; ideal—BMI from 18.5 to 25 kg/m²; overweight—BMI from 25 to 30 kg/m²; obese—BMI from 30 to 40 kg/m²; morbidly obese—BMI greater than 40 kg/m².

In some embodiments, the method of the present invention is particularly suitable for inhibiting the lipolysis of white adipose tissue, and the loss of skeletal muscle. In some embodiments, the method of the present invention is particularly suitable for stimulating appetite.

Underweight may be due to several causes, such as rapid metabolism, poor/inadequate diet or starvation (malnutrition), malabsorption due to defective intestinal function, endocrine disturbances e.g. type I diabetes, psychological problems (such as anorexia nervosa, body dysmorphic disorder, stress and anxiety) and weight loss, due to chronic illnesses and ageing. While in general the underlying cause of the underweight will have to be treated per se, the underweight too may be a health hazard, and as such have to be treated in itself. Indeed, persons suffering from underweight generally have poor physical stamina, a weakened immune system, as well as being at higher risk of developing diseases such as osteoporosis, heart disease and vascular disease. Additionally, in the female sex, underweight can lead to delayed sexual development, retarded amenorrhoea or complications during pregnancy.

In some embodiments, the subject suffers from a wasting disorder. As used herein, the term “wasting disorder” has its general meaning in the art and includes but is not limited to anorexia cachexia, anorexia of the aged, anorexia nervosa, cachexia associated with cancer, cachexia associated with AIDS, cachexia associated with heart failure, cachexia associated with cystic fibrosis, cachexia associated with rheumatoid arthritis, cachexia associated with kidney disease, cachexia associated with chronic obstructive pulmonary disease (COPD), cachexia associated with ALS, cachexia associated with renal failure or cachexia associated, and other disorders associated with aberrant appetite, fat mass, energy balance, and/or involuntary weight loss.

In some embodiments, the subject suffers from “cachexia”. As used herein, the term “cachexia” is used for a condition of physical wasting with loss of body fat and muscle mass. Generally, cachexia may be associated with and due to conditions such as cancer, required immunodeficiency syndrome (AIDS), cardiac diseases, infectious diseases, shock, burn, endotoxinemia, organ inflammation, surgery, diabetes, collagen diseases, radiotherapy, and chemotherapy. In many of these diseases, cachexia may significantly contribute to morbidity or mortality. Another particular group of individuals that are susceptible to developing a cachectic state are those individuals that have undergone a gastrectomy, such as may be practiced on gastric cancer and ulcer patients.

In some embodiments, the subject suffers from anorexia. As used herein, the term “anorexia” has its general meaning in the art and refers to any eating disorder characterized by markedly reduced appetite or total aversion to food. In some embodiments, the subject suffers from anorexia nervosa. In general, subjects suffering from anorexia nervosa have a BMI of less than 17.5 kg/m2.

Accordingly, the present invention is drawn to methods of treating a patient exhibiting one or more wasting disorders such as anorexia, cachexia, anorexia of the aged, anorexia nervosa, cachexia associated with cancer, cachexia associated with AIDS, cachexia associated with heart failure, cachexia associated with cystic fibrosis, cachexia associated with rheumatoid arthritis, cachexia associated with kidney disease, cachexia associated with COPD, cachexia associated with ALS, cachexia associated with renal failure or cachexia associated, or hip fracture, and in reducing the mortality and morbidity of critically ill patients, comprising administering to said patient in need of such treatment a therapeutically effective of an inhibitor of NTSR1 activation or expression.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

In some embodiments, the method of the present invention is particularly suitable for treating cachexia in a subject suffering from cancer. As used herein, the term “cancer” has its general meaning in the art and includes, but is not limited to, solid tumors and blood borne tumors. The term cancer includes diseases of the skin, tissues, organs, bone, cartilage, blood and vessels. The term “cancer” further encompasses both primary and metastatic cancers. Examples of cancers that may treated by methods and compositions of the invention include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; leydig cell tumor, malignant; lipid cell tumor, malignant; paraganglioma, malignant; extra-mammary paraganglioma, malignant; pheochromocytoma; glomangio sarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

As used herein, the term “NTSR1” has its general meaning in the art and refers to neurotensin receptor 1 (Gene ID: 4923) which belongs to the large superfamily of G-protein coupled receptors. The natural ligands of NTSR1 is neurotensin (NTS) or “neuromedin N”.

As used herein, the term “inhibitor of NTSR1 activation or expression” should be understood broadly, this expression refers to agents down-regulating the expression of neurotensin or neuromedin N or NTSR1, compounds that bind to neurotensin or to neuromedin N or to NTSR1 and inhibit the neurotensin activation of NTSR1 or the neuromedin N activation of NTSR1, or a protease that can degrade NTS or neuromedin N.

Examples of inhibitors of NTSR1 activation or expression may be selected from the group consisting of an agent down-regulating the expression of NTS or the expression of neuromedin N or the expression of NTSR1, an antibody against NTS or an antibody against neuromedin N, a fragment thereof which binds to NTS or a fragment thereof which binds to neuromedin N, an antibody against the NTSR1 or a fragment thereof which binds to the NTSR1, and an antagonist of the NTSR1.

As used herein, the term “neurotensin” (NTS) has its general meaning in the art and refers to a 13-amino acid peptide that is produced from a single precursor, pre-pro-neurotensin, which gives rise to both NTS and the related peptide neuromedin N. The term encompasses both short and long fragments. According to the present invention, the short fragment of neurotensin is represented by SEQ ID NO: 1 and the long fragment is represented by SEQ ID NO: 2.

SEQ ID NO: 1
QLYENKPRRP YIL
SEQ ID NO: 2

| | |
|---|---|
| 1 | MMAGMKIQLV CMLLLAFSSW SLCSDSEEEM KALEADFLTN |
| | MHTSKISKAH VPSWKMTLLN |
| 61 | VCSLVNNLNS PAEETGEVHE EELVARRKLP TALDGFSLEA |
| | MLTIYQLHKI CHSRAFQHWE |
| 121 | LIQEDILDTG NDKNGKEEVI KRKIPYILKR QLYENKPRRP |
| | YILKRDSYYY |

As used herein, the term “neuromedin N” has its general meaning in the art and refers to a 6-amino acid peptide. The term encompasses both short and long fragments. According to the present invention, the short fragment of neuromedin N is represented by SEQ ID NO: 19 and the long fragment is represented by SEQ ID NO: 20

SEQ ID NO: 19
KIPYIL
SEQ ID NO: 20

| | |
|---|---|
| 1 | MMAGMKIQLV CMLLLAFSSW SLCSDSEEEM KALEADFLTN |
| | MHTSKISKAH VPSWKMTLLN |
| 61 | VCSLVNNLNS PAEETGEVHE EELVARRKLP TALDGFSLEA |
| | MLTIYQLHKI CHSRAFQHWE |
| 121 | LIQEDILDTG NDKNGKEEVI KRKIPYILKR |

As used herein, the term “antibody” is thus used to refer to any antibody-like molecule that has an antigen binding region, and this term includes antibody fragments that comprise an antigen binding domain such as Fab′, Fab, or F(ab′)2. The techniques for preparing and using various antibody-based constructs and fragments are well known in the art. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Antibodies and antibody-fragments can be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments.

In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CHI, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDR set from each of a heavy and a light chain V region. Framework Regions (FRs) refer to amino acid sequences interposed between CDRs. The residues in antibody variable domains are conventionally numbered according to a system devised by Kabat et al. This system is set forth in Kabat et al., 1987, in Sequences of Proteins of Immunological Interest, US Department of Health and Human Services, NIH, USA (hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35B (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

As used herein the term “bind” indicates that the antibody has affinity for the surface molecule. The term “affinity”, as used herein, means the strength of the binding of an antibody to an epitope. The affinity of an antibody is given by the dissociation constant Kd, defined as [Ab]×[Ag]/[Ab-Ag], where [Ab-Ag] is the molar concentration of the antibody-antigen complex, [Ab] is the molar concentration of the unbound antibody and [Ag] is the molar concentration of the unbound antigen. The affinity constant Ka is defined by 1/Kd. Preferred methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of mAbs is the use of Biacore instruments.

In some embodiments, the antibody is a humanized antibody. As used herein, “humanized” describes antibodies wherein some, most or all of the amino acids outside the CDR regions are replaced with corresponding amino acids derived from human immunoglobulin molecules. Methods of humanization include, but are not limited to, those described in U.S. Pat. Nos. 4,816,567, 5,225,539, 5,585,089, 5,693,761, 5,693,762 and 5,859,205, which are hereby incorporated by reference.

In some embodiments, the antibody is a fully human antibody. Fully human monoclonal antibodies also can be prepared by immunizing mice transgenic for large portions of human immunoglobulin heavy and light chain loci. See, e.g., U.S. Pat. Nos. 5,591,669, 5,598,369, 5,545,806, 5,545,807, 6,150,584, and references cited therein, the contents of which are incorporated herein by reference.

In some embodiments, the antibody of the present invention is a single chain antibody. As used herein the term “single domain antibody” has its general meaning in the art and refers to the single heavy chain variable domain of antibodies of the type that can be found in Camelid mammals which are naturally devoid of light chains. Such single domain antibody are also “nanobody®”. For a general description of (single) domain antibodies, reference is also made to the prior art cited above, as well as to EP 0 368 684, 17); and WO 06/030220, WO 06/003388.

According to the present invention, the antibody is a neutralizing antibody. As used herein, the term “neutralising antibody” describes an antibody that is capable of neutralising the biological activity of neurotensin for example by blocking binding of neurotensin to their corresponding receptors. The neutralising antibody of the present invention is also capable of neutralizing the biological activity of neuromedin N for example by blocking binding of .neuromedin N to their corresponding receptors. It will be appreciated that the term “neutralizing” as used herein refers to a reduction in biological activity which may be partial or complete.

In some embodiments, the antibody comprises heavy and light chain variable regions of an antibody designated NTSp27.7.4. The heavy chain variable region of NTSp27.7.4 has the amino acid as set forth in SEQ ID NO:3, and the light chain variable region of NTSp27.7.4 the amino acid sequence as set forth in SEQ ID NO:4.

(Heavy Chain of of NTSp27.7.4)
SEQ ID NO: 3
DVKLVESGGGLVKLGGSLKLSCAASGFTFSGYYMSWVRQTPEKRLELVAA
INNYGDNTNYPDTVKGRFSVSRDNAKNTLYLEMNSLKSEDTALYYCARLA
NYANQRGAMDYWGQGTSVTVSS
(Light chain of of NTSp27.7.4)
SEQ ID NO: 4
DIQMTHTTSSLSASLGDRVTISCRASQDIANYLNWYQQKPDGTVTLLIYY
TSRLHSGVPSRFSGSGSGTDYSLTINNLDQEDIATYFCQQGYTLPPTFGG
GTKLEIK

In some embodiments, the antibody comprises heavy and light chain variable regions of an antibody designated FLp26-8.2. The heavy chain variable region of FLp26-8.2 has the amino acid as set forth in SEQ ID NO:5, and the light chain variable region of FLp26-8.2 the amino acid sequence as set forth in SEQ ID NO: 6.

(heavy chain of FLp26-8.2):
SEQ ID NO: 5
QIQLVQSGPELICKPGETVKISCKASGYTFTNYGMNWVKQAPGKGLKWMG
WITTNTGEPTYAEEFKGRFAFSLETSASTAYLQINNLKNEDTATYFCARR
AFAMDYWGQGTSVTVSS
(light chain of FLp26-8.2):
SEQ ID NO: 6
DIVMTQAAPSVPVTPGESVSISCRSSKSLLHSNGNTYLYWFLQRPGQSPQ
LLIYRMSNLASGVPDRFSGSGSGTAFTLRISRVEAEDVGVYYCMQHLEYP
YTFGGGTKLEIK

In some embodiments, the antibody contains the heavy chain CDRs of the heavy chain variable region of NTSp27.7.4 (SEQ ID NO: 3) or FLp26-8.2 (SEQ ID NO: 4), respectively represented by SEQ ID NO: 7-9 and SEQ ID NO: 13-15. In some embodiments, the antibody of the present invention comprises the light chain CDRs of the light chain variable region of NTSp27.7.4 (SEQ ID NO: 5) or FLp26-8.2 (SEQ ID NO: 6) respectively represented by SEQ ID NO: 10-12 and SEQ ID NO: 16-18.

In some embodiments, the antibody comprises the heavy chain CDRs of the heavy chain variable region of NTSp27.7.4 (SEQ ID NO: 3) and the light chain CDRs of the light chain variable region of NTSp27.7.4 (SEQ ID NO: 4).

In some embodiments, the antibody comprises the heavy chain CDRs of the heavy chain variable region of FLp26-8.2 (SEQ ID NO: 5) and the light chain CDRs of the light chain variable region of FLp26-8.2 (SEQ ID NO: 6).

| | (H-CDR1 of NTSp27.7.4) |
|---|---|
| SEQ ID NO: 7 | |
| | GFTFSGYYMS |
| | (H-CDR2 of NTSp27.7.4) |
| SEQ ID NO: 8 | |
| | AINNYGDNTNYPDTVKG |
| | (H-CDR3 of NTSp27.7.4) |
| SEQ ID NO: 9 | |
| | LANYANQRGAMDY |
| | (L-CDR1 of NTSp27.7.4) |
| SEQ ID NO: 10 | |
| | RASQDIANYLN |
| | (L-CDR2 of NTSp27.7.4) |
| SEQ ID NO: 11 | |
| | YTSRLHS |
| | (L-CDR3 of NTSp27.7.4) |
| SEQ ID NO: 12 | |
| | QQGYTLPPT |
| | (H-CDR1 FLp26-8.2) |
| SEQ ID NO: 13 | |
| | GYTFTNYGMN |
| | (H-CDR2 FLp26-8.2) |
| SEQ ID NO: 14 | |
| | WITTNTGEPTYAEEFKG |
| | (H-CDR3 FLp26-8.2) |
| SEQ ID NO: 15 | |
| | RAFAMDY |
| | (L-CDR1 FLp26-8.2) |
| SEQ ID NO: 16 | |
| | RSSKSLLHSNGNTYLY |
| | (L-CDR2 FLp26-8.2) |
| SEQ ID NO: 17 | |
| | RMSNLAS |
| | (L-CDR3 FLp26-8.2) |
| SEQ ID NO: 18 | |
| | MQHLEYPYT |

In some embodiments, the antibody of the present invention is an antibody against NTS or an antibody against neuromedin N.

As used herein, the term “epitope” refers to a specific arrangement of amino acids located on a protein or proteins to which an antibody binds. Epitopes often consist of a chemically active surface grouping of molecules such as amino acids or sugar side chains, and have specific three-dimensional structural characteristics as well as specific charge characteristics. Epitopes can be linear or conformational, i.e., involving two or more sequences of amino acids in various regions of the antigen that may not necessarily be contiguous.

In some embodiment, the present invention relates to an antibody that binds to an epitope comprising amino acid residues from amino acid residues 123 to 137 of SEQ ID NO: 2 or of SEQ ID NO: 20.

In some embodiment, the present invention relates to an antibody that binds to an epitope comprising 1, 2, 3, 4, 5, 6, 7, 8, 9, 10, 11, 12, 13, 14, 15 amino acid residues from amino acid residues 123 to 137 of SEQ ID NO: 2 or of SEQ ID NO: 20.

In some embodiments, the antibody of the invention binds to an epitope comprising amino acid residues from amino acid residues 123 to 137 of SEQ ID NO: 2 or of SEQ ID NO: 20.

In some embodiments, the antibody of the invention binds to an epitope comprising 1, 2, 3, 4, 5, 6, 7, 8, 9, 10, 11, 12, 13, 14, 15 amino acid residues from amino acid residues 123 to 137 of SEQ ID NO: 2 or of SEQ ID NO: 20.

In some embodiments, the antibody of the invention binds to a conformational epitope.

Additional competing antibodies can be identified based on their ability to cross-compete (e.g., to competitively inhibit the binding of, in a statistically significant manner) with the antibody of the invention in standard binding assays. The ability of a test competing antibody to inhibit the binding of antibody of the present invention to an epitope comprising amino acid residues from amino acid residues 123 to 137 of SEQ ID NO: 2 or of SEQ ID NO: 20 demonstrates that the test competing antibody can compete with the antibody of the invention for binding to an epitope comprising amino acid residues from amino acid residues 123 to 137 of SEQ ID NO: 2 or of SEQ ID NO: 20; such an competing antibody may, according to non-limiting theory, bind to the same or a related (e.g., a structurally similar or spatially proximal) epitope as the antibody of the invention with which it competes. Thus, another aspect of the invention provides a competing antibody that bind to the same antigen as, and compete with, the antibody of the invention disclosed herein. Cross-competition is present if antibody A reduces binding of antibody B at least by 60%, specifically at least by 70% and more specifically at least by 80% and vice versa in comparison to the positive control which lacks one of said antibodies. As the skilled artisan appreciates competition may be assessed in different assay set-ups. One suitable assay involves the use of the Biacore technology (e.g., by using the BIAcore 3000 instrument (Biacore, Uppsala, Sweden)), which can measure the extent of interactions using surface plasmon resonance technology. Another assay for measuring cross-competition uses an ELISA-based approach. Furthermore, a high throughput process for “binning” antibodies based upon their cross-competition is described in International Patent Application No. WO2003/48731.

As used herein, a competing antibody “competes” for binding when the competing antibody inhibits epitope comprising amino acid residues from amino acid residues 123 to 137 of SEQ ID NO: 2 or of SEQ ID NO: 20 binding of the antibody of the invention or antigen binding fragment of the invention by more than 50,51,52,53,54,55,56,57,58,59,60,61,62,63,64,65,66,67,68,69,70,71,72,73,74,75,76,77,78,79, 80,81,82,83,84,85,86,87,88,89,90,91,92,93,94,95,96,97,98 or 99% in the presence of an equimolar concentration of competing antibody.

In some embodiments, a competing antibody cross-competes for binding to the epitope comprising amino acid residues from amino acid residues 123 to 137 of SEQ ID NO: 2 or of SEQ ID NO: 20 with the monoclonal antibody of the invention comprising a heavy chain comprising the following CDR: i) the H-CDR1 of NTSp27.7.4 as set forth in SEQ ID NO: 7, ii) the H-CDR2 of NTSp27.7.4 as set forth in SEQ ID NO: 8 and iii) the H-CDR3 of NTSp27.7.4 as set forth in SEQ ID NO: 9 and a light chain comprising the following CDR i) the L-CDR1 of NTSp27.7.4 as set forth in SEQ ID NO 10, ii) the L-CDR2 of NTSp27.7.4 as set forth in SEQ ID NO: 11 and iii) the L-CDR3 of NTSp27.7.4 as set forth in SEQ ID NO: 12.

In some embodiments, a competing antibody cross-competes for binding to the epitope comprising amino acid residues from amino acid residues 123 to 137 of SEQ ID NO: 2 or of SEQ ID NO: 20 with the monoclonal antibody of the invention comprising a heavy chain comprising the following CDR: i) the H-CDR1 FLp26-8.2 as set forth in SEQ ID NO: 13, ii) the H-CDR2 FLp26-8.2 as set forth in SEQ ID NO: 14 and iii) the H-CDR3 FLp26-8.2 as set forth in SEQ ID NO:15 and light chain comprising the following CDR i) the L-CDR1 FLp26-8.2 as set forth in SEQ ID NO:16, ii) the L-CDR2 FLp26-8.2 as set forth in SEQ ID NO:17 and iii) the L-CDR3 FLp26-8.2 as set forth in SEQ ID NO:18.

According to the present invention, the cross-competing antibody as above described retain the activity of the monoclonal antibody of the invention which comprises a heavy chain comprising the following CDR: i) the H-CDR1 of NTSp27.7.4 as set forth in SEQ ID NO: 7, ii) the H-CDR2 of NTSp27.7.4 as set forth in SEQ ID NO: 8 and iii) the H-CDR3 of NTSp27.7.4 as set forth in SEQ ID NO: 9 and a light chain comprising the following CDR i) the L-CDR1 of NTSp27.7.4 as set forth in SEQ ID NO 10, ii) the L-CDR2 of NTSp27.7.4 as set forth in SEQ ID NO: 11 and iii) the L-CDR3 of NTSp27.7.4 as set forth in SEQ ID NO: 12.

According to the present invention, the cross-competing antibody as above described retain the activity of the monoclonal antibody of the invention which comprises a heavy chain comprising the following CDR: i) the H-CDR1 FLp26-8.2 as set forth in SEQ ID NO: 13, ii) the H-CDR2 FLp26-8.2 as set forth in SEQ ID NO: 14 and iii) the H-CDR3 FLp26-8.2 as set forth in SEQ ID NO:15 and light chain comprising the following CDR i) the L-CDR1 FLp26-8.2 as set forth in SEQ ID NO:16, ii) the L-CDR2 FLp26-8.2 as set forth in SEQ ID NO:17 and iii) the L-CDR3 FLp26-8.2 as set forth in SEQ ID NO:18.

By a “therapeutically effective amount” is meant a sufficient amount of the antibody of the present invention for reaching a therapeutic effect. It will be understood, however, that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 4,000 mg per adult per day. Typically, the compositions contain 0.01, 0.05, 0.1, 0.5, 1.0, 2.5, 5.0, 10.0, 15.0, 25.0, 50.0, 100, 250, 500 and 1000 mg of the active ingredient for the symptomatic adjustment of the dosage to the subject to be treated. A medicament typically contains from about 0.01 mg to about 1000 mg of the active ingredient. An effective amount of the drug is ordinarily supplied at a dosage level from 0.0002 mg/kg to about 50 mg/kg of body weight per day, especially from about 0.001 mg/kg to 10 mg/kg of body weight per day.

The antibody of the present invention is administered to the subject in a form of a pharmaceutical composition. Typically, the antibody of the present invention can be combined with pharmaceutically acceptable excipients, and optionally sustained-release matrices, such as biodegradable polymers, to form therapeutic compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to the subjects. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the present invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The antibody of the present invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The preparation of more, or highly concentrated solutions for direct injection is also contemplated, where the use of DMSO as solvent is envisioned to result in extremely rapid penetration, delivering high concentrations of the active agents to a small tumor area. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

The invention will be further illustrated by the following figures and examples. However, these examples and figures should not be interpreted in any way as limiting the scope of the present invention.

### FIGURES

FIG. 1. LF-NTS mAb treatments induced a gain of weight under regular diet. (A) Weight of mice fed with chow treated or not once a weeks with PBS, or 5 mg/kg i.v. LF-NTS mAb n=10 In a two-way ANOVA statistical analysis, was P<0.05 from day 145 to day 247. Inset Graph displaying the data from day 1 to 29. (B) Percentage of weight gained from day 1 by the mouse over time. In t test was p<0.05 from day 150 to day 248.

FIG. 2. LF-NTS mAb treatment induced an increase in blood albumin level. Ratio albumin/total protein from blood taking on fasted mice for 6 h at day 72 (n=6) comparison between PBS and LF-NTS mAb treated animals. In t test*p<0.05

FIG. 3. LF-NTS mAb treatment induced an increase mouse physical activity. Mouse activity recorded from individual mouse during 48 h (ADDENFI, Les Cordeliers, Paris, France) at day 63 (A) and day 125 (B). At D300, activity with recorded with an actimeter (Immetronic, Z, France), horizontal activity (C) and rearing (D) were recorded. Recording performed on PBS or LF-NTS mAb treated animals at day 63 (n=6), d125 (n=8), and d300 (n=8) was split in three time period. Int test *p<0.05, **p<0.01, ***p<0.001, ****p<0.0001

FIG. 4. LF-NTS mAb treatment induced an increase the weight of the muscle. At the sacrifice the tibialis (A) and gastrocnemius (B) were dissected and weight (n=9).

FIG. 5. LF-NTS mAb treatment induced an increase the weight of the major organs. At the sacrifice the Liver (A), spleen (B), pancreas (C) and Inguinal white adipose tissue (D) were dissected and weight (n=9).

FIG. 6. LF-NTS mAb treatments induced a gain of weight under high fat diet. (A) Weight of mice fed with 60% high fat diet (HFD) treated or not once a weeks with PBS or 5 mg/kg i.v. LF-NTS mAb n=5. In a two-way ANOVA statistical analysis *p<0.05 (B) Percentage of weight gained from day 1 by the mouse over time. In t test p<0.05 at day 75 and 100;

FIG. 7. LF-NTS mAb treatments induced an increase in food intake. The weight of HFD eaten per cage of 5 animals was calculated. The graph represents the weight of food eaten per week and per animal treated or not with LF-NTS mAb.

FIG. 8. LF-NTS mAb treatment induced an increase in blood protein and albumin level. Level of albumin or protein from blood taking on fasted mice for 6 h at day 110 (n=5) fed with HFD, and treated or not with LF-NTS mAb. In t test *p<0.05

FIG. 9. LF-NTS mAb treatment induced an increase mouse physical activity. (A-B) Mouse activity recorded from individual mouse during 48 h (ADDENFI, Les Cordeliers, Paris, france). Recording was split in three time period, the recording was performed at day 53 and day 120 (n=5) of HFD on animals treated with PBS or LF-NTS mAb. (C) Rearing was recorded with an actimeter (Immetronic, France) over a period of 48 h at day 110 on another set of mice with an average weight of 50 g. Int test *p<0.05, **p<0.01, ***p<0.001,

FIG. 10. LF-NTS mAb treatment induced an increase of the muscle fibers surface. At day 130 after HFD, (A) calculation of gastrocnemius muscle fibers surface performed on 200 fibers (n=7) (B) calculation of tibialis muscle fibers surface performed on 150 fibers (n=6). (C) and (D) Distribution of the muscle fibre size of gastrocnemius or tibialis, respectively, on animal treated or not with LF-NTS mAb. In t test ****p<0.0001.

FIG. 11. LF-NTS mAb prevent the cachexia induced by cancer. (A) LF-NTS mAb treatment decrease the tumor growth rate of mouse lung carcinoma sub-clone (LLC1-A1) n=10 In 2way Anova *p<0.05. (B) Calculation of the percentage of weight (body without tumor) as compared to the weight at day 1 (day of grafting). In t test *p<0.05. (C) Weight (g) of the carcass (animals without organs, with skin, bones muscles, and head). In t test one tail *p<0.05.

FIG. 12. LF-NTS mAb treatments prevent the emptying of epididymal white adipose tissues. (A) LF-NTS mAb treatment reduced the weight loss of epididymal white adipose tissues induced by cachexia n=9. In t test *p<0.05 **p<0.01. (B) Size of epididymal white adipocytes from mice nonbearing tumor, mice bearing tumor treated with PBS or LF NTS mAb. In t test *p<0.05 ***p<0.001. (C) Frequency distribution (percentage) of the epididymal white adipocytes from nonbearing tumor mice, or mice bearing tumor treated with PBS or with LF NTS mAb. (n=7)

FIG. 13. LF-NTS mAb treatments prevent the emptying of retroperitoneal white adipose tissues. (A) LF-NTS mAb treatment reduced the weight loss of retroperitoneal white adipose tissues induced by cachexia n=9. In t test *p<0.05 **p<0.01. (B) Size of retroperitoneal white adipocytes from nonbearing tumor mice, tumor bearing mice treated with PBS or LF NTS mAb, Int test *p<0.05 **p<0.01. (C) Frequency distribution (percentage) of the retroperitoneal white adipocyte from nonbearing tumor mice, tumor bearing mice treated with PBS or LF NTS mAb (n=6).

### EXAMPLE

### Material & Methods

Animals

Male C57BL/6j mice were purchased at 4 or 3 weeks old from (Janvier™) after a week of acclimation mice were separated in two groups and treated once a week i.v. with PBS or 5 mg/kg for 247 days. Mice were fed by regular chow, LASQCdiet ROD-16R LAS vendi or by high fat diet (HFD) 14.6% protein, 58.8% fat, and 26;7% carbohydrate from SAFE (ref 260 HF). All the procedures were in accordance with the “Guide of the Care and Use of laboratory Animals”. Institutional Review Board approval was obtained by «Le Comité d'Ethique en l'Expérimentation Animale Charles Darwin # B751201».

Plasma Biochemical Analysis.

Total protein and albumin were determined using a benchtop biochemistry analyzer according to the manufacturers' protocol (Randox Laboratories Ltd, Roissy en France, France).

Activity

Global activity was measured on an activity platform to measure the (ADDENFI, Les Cordeliers, Paris, France). Mice were individually housed in a modular chamber (45×35 cm) surrounded by 50 cm-high walls. In this system, the floor plate recover with litter is resting on piezoelectric pressure sensors, providing continuous analog signal generated by the subtle changes in floor-plate pressure due to animal movement. Signals were analysed by Alab suite fida software.

Rearing was measured in an actimeter (Immetronic, France) composed of eight cages (19×11×14 cm) under low illumination (<5 lx). One mouse was placed in each box and its displacements were measured by photocell beams located across the long axis and above the floor. Rearing activity was recorded during 36 h and expressed in counts/12 h as the total number of interruption of the photocell beams.

Muscular Fibers and Adipocytes Surface Calculation

After dissection, the muscle and adipocytes were fixed with 4% paraformaldehyde, and embedded in paraffin wax. Standard haematoxylin and eosin staining was performed. Surface calculation was performed using Image J software

Tumors

Eight-week-old male athymic NMRI-Foxn1nu/nu mice (Janvier™) and C57BL/6j (Janvier™) were used. Mice were injected in the flanks with 0.25×10⁶ of mouse lung carcinoma cells, LLC1 subclone A1, and allowed to grow until tumors reached a volume of 35 to 40 mm³ (tumor volume were calculated using ellipsoid formula). Animals were then randomized. Two groups of 10 mice were formed and treated by intra-orbital injections with LF NTS mAb (5 mg/kg once a week), or vehicle (PBS).

Results

Effect of LF-NTS mAb on Mice Fed with Normal Diet.

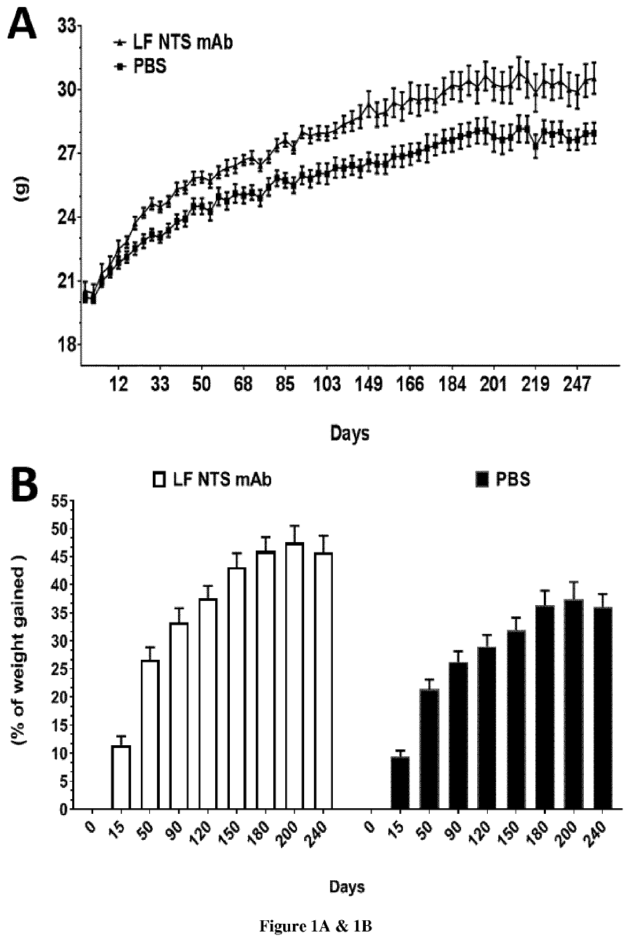

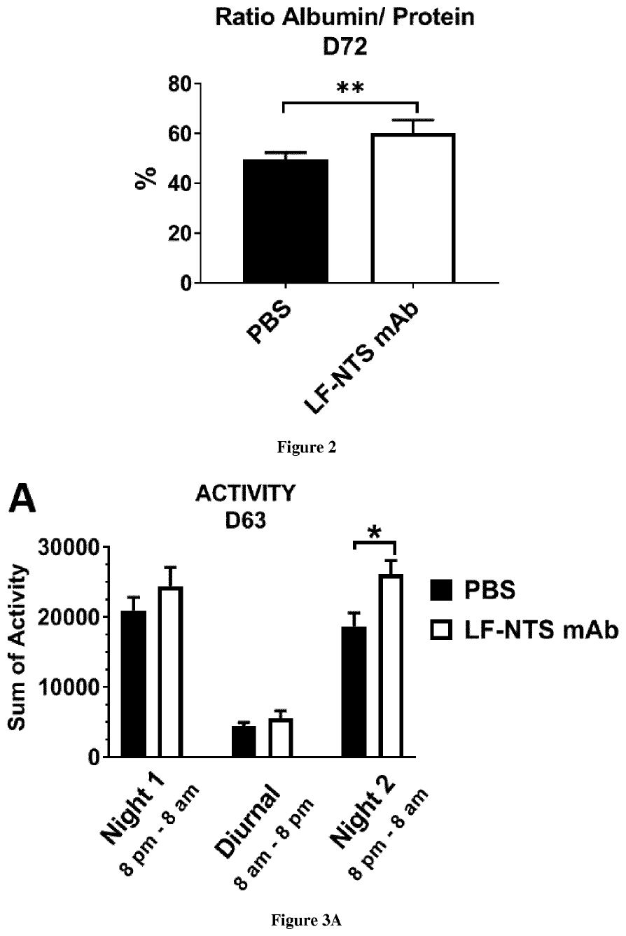

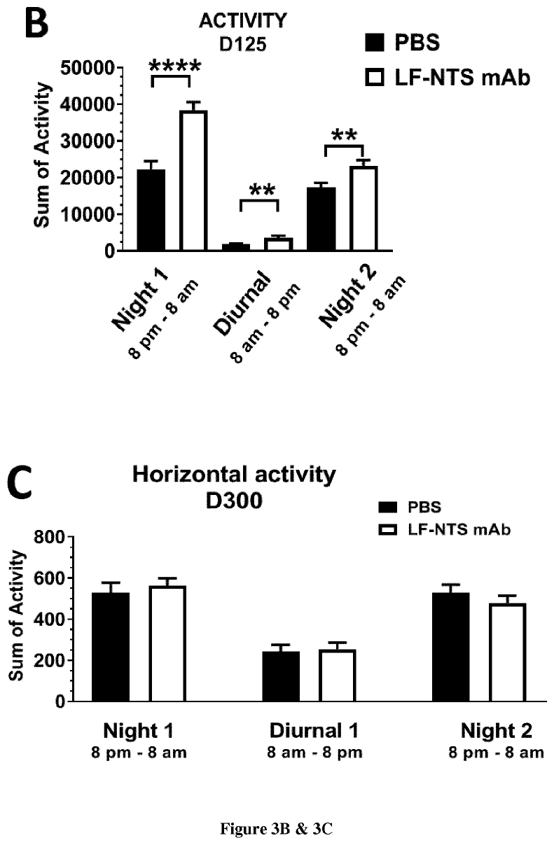

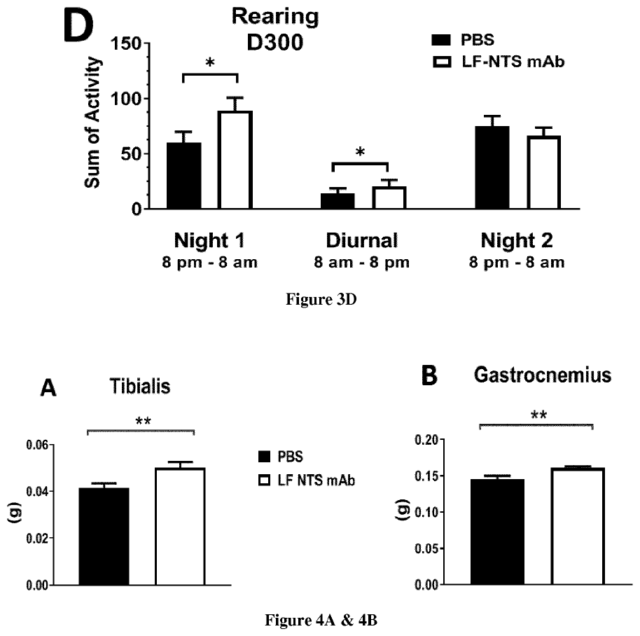

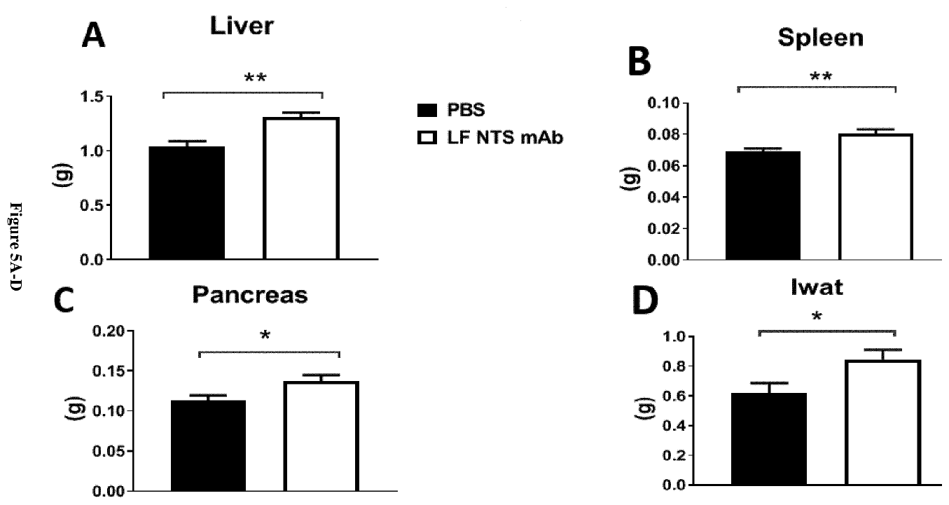

Effect of LF-NTS mAb treatment was evaluated on C57BL/6j mice treated once a week with 5 mg/kg, or PBS for 247 days, and under normal diet. The first injection was performed when the mice were 5 weeks old. As seen on FIG. 1A the mice treated with NTS mAb put on weight along the treatment as compared to control mice. The percentage of weight gained was calculated from day 1. The inset graph shows that the gain of weight start just after the first injection and was significant at day 24. The FIG. 1B shows that the percentage of gained weight is increasing with time. The average increase rate was of 22%. No difference in food intake was observed (Data not shown). Basic blood parameters, glucose cholesterol, and triglyceride levels were not modified by the treatment (Data not shown). The weight of feces collected during 48 h was equivalent (Data not shown). The ratio albumin to protein was significant 17% higher in LF-NTS mAb treated mice as compared to control (FIG. 2). Mice treated with LF-NTS mAb are more active than PBS treated mice, this increase and persist with time. At day 63 and 125 the global activity was measured with the ADDENFI cage (FIGS. 3A and 3B) At day 300 the actimeter measurement revealed that rearing was preferentially increased (FIGS. 3C and 3D). At the sacrifice, the weight of muscle, tibialis and gastrocnemius (FIGS. 4A and 4B), as well as the major organs, liver, pancreas, spleen, and inguinal white adipose tissue (Iwat) was also increased with the LF-NTS mAb (FIGS. 5A to 5D)

Effect of LF-NTS mAb on Mice Fed with High Fat Diet.

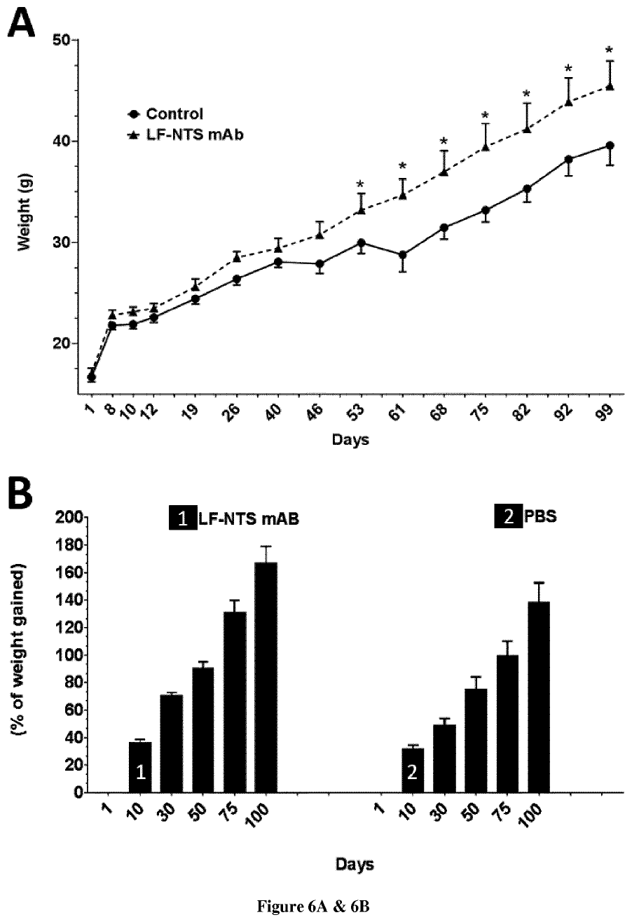

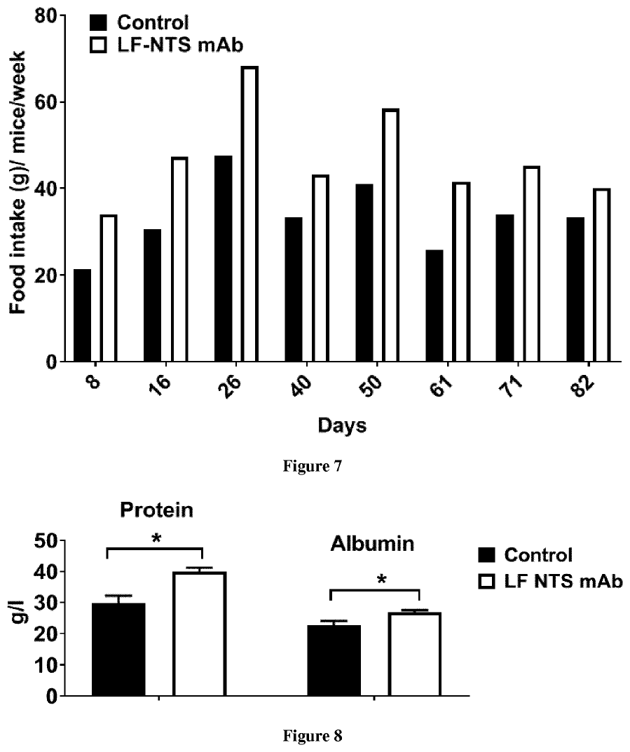

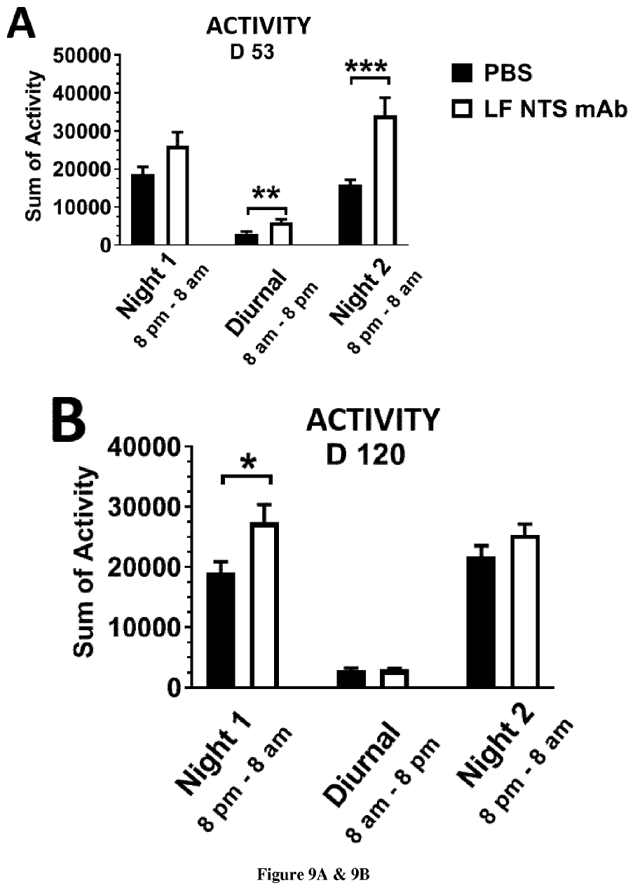

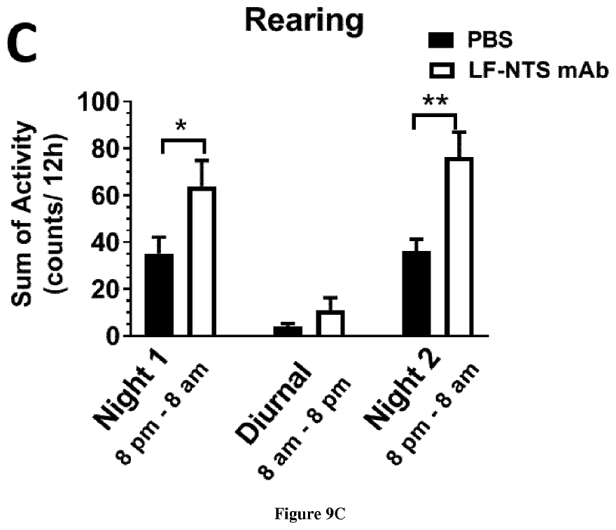

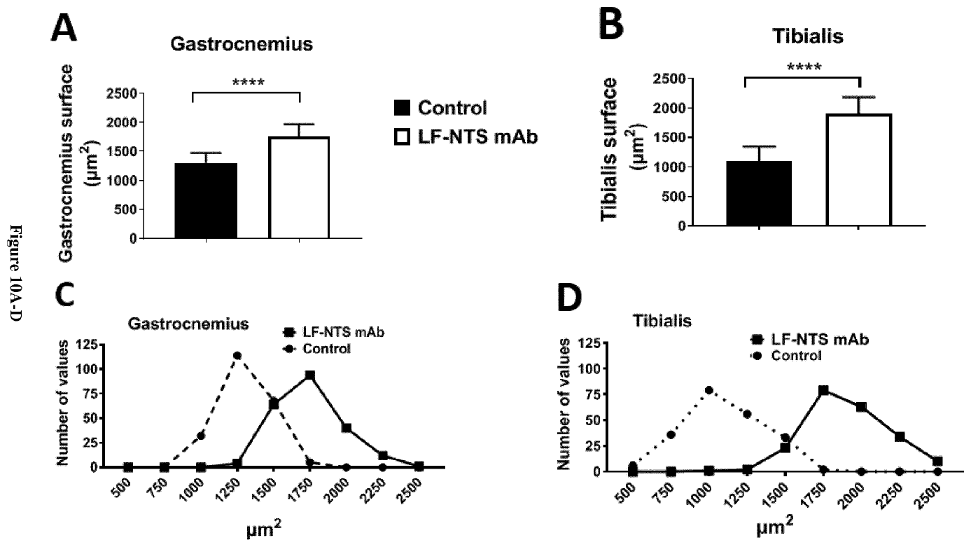

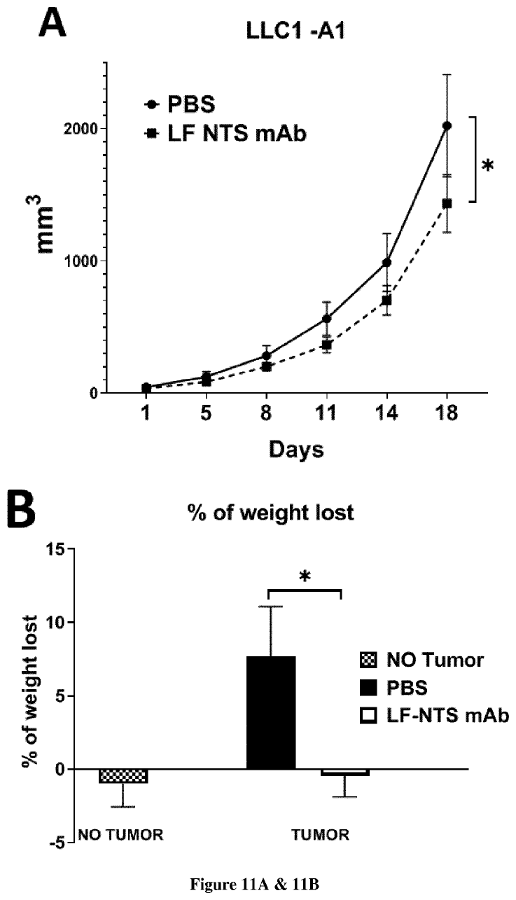

Similar experiment was performed on C57BL/6j mice treated once a week with 5 mg/kg, or PBS for 120 days, and under high fat diet. After weaning the mice were on chow for a week and at 4 week old mice the food was switch to HFD. The first injection was performed two days before the diet switch. As seen previously the mice treated with NTS mAb put on weight along with the treatment as compared to control mice (FIGS. 6A & 6B), the percentage of gained weight is increasing with time the average increase rate was of 19%. Food intake was evaluated every 10 days, mice treated LF-NTS mAb ate 30 to 50% more than the control mice (FIG. 7). Blood sample taken after 110 day of diet and treatment showed that protein and albumin were 34 and 18% higher respectively when mice were treated with antibody (FIG. 8). Finally, the mice under NTS-LF mAb were also more active as shown in FIG. 9, in particular the exploratory behavior induced by novelty, rearing was strongly increased in mice treated with mAb (FIGS. 9A to 9C). This more active behavior affects muscle fibers size of the gastrocnemius and the tibialis. The sire of the fibers was 34 and 110% higher in the gastrocnemius and the tibialis, respectively (FIGS. 10A and 10B). The distribution of the fiber sizes shown in FIGS. 10C and 10D show that globally all the muscle fibers have grown; the small fibers have almost disappeared in the animals treated with the antibody.

Effect of LF-NTS mAb on Tumor Bearing Mice

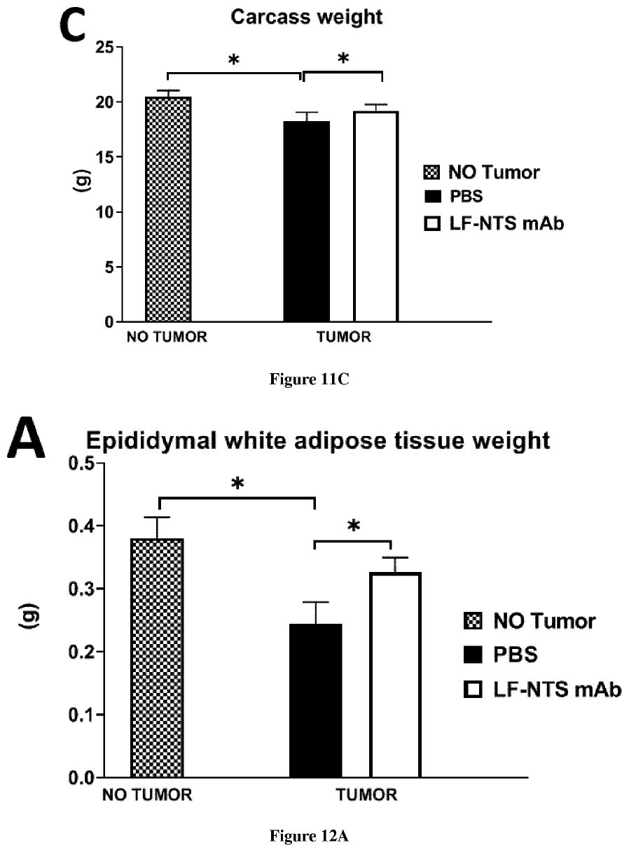

We tested the LF-NTS antibody on tumor growth of LLC1-A1 sub-clone. We observed a diminution of the tumor growth rate (FIG. 11A). Eighteen days after grafting the animals were sacrificed and weighed. The tumor bearing animals lost 7.67±3.4% of their weight as compared to the weight before grafting. This weight lost was not observed in the mice-bearing tumor and treated with LF-NTS mAb, or mice with no tumor (FIG. 11B). The weight of the carcass was also diminished when mice were treated with PBS, as compared to mice treated with LF NTS mAb, or mice not bearing tumors (FIG. 11C). The LLC1-A1 cells grafted in mice induced cachexia and the LF NTS mAb inhibited the weight lost.

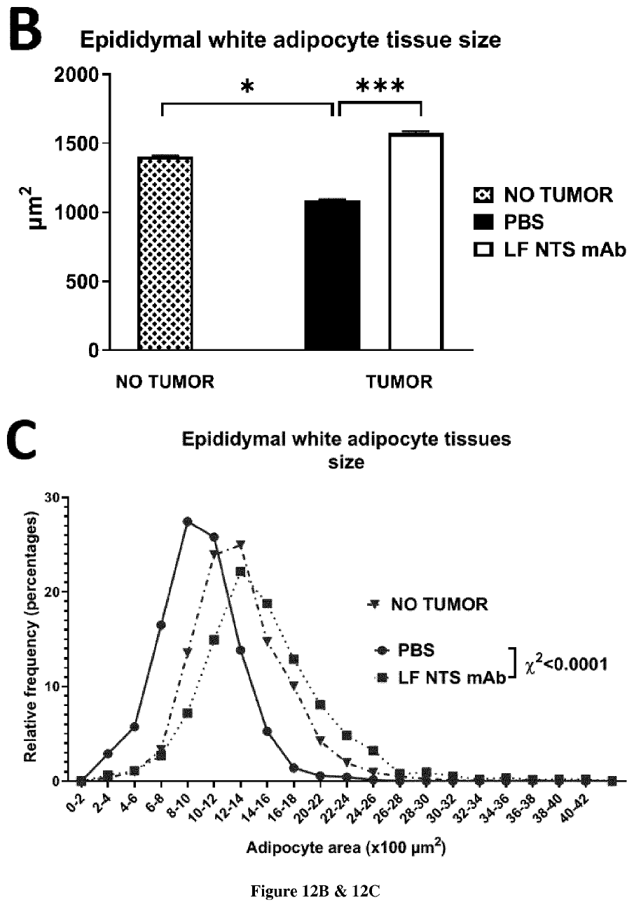

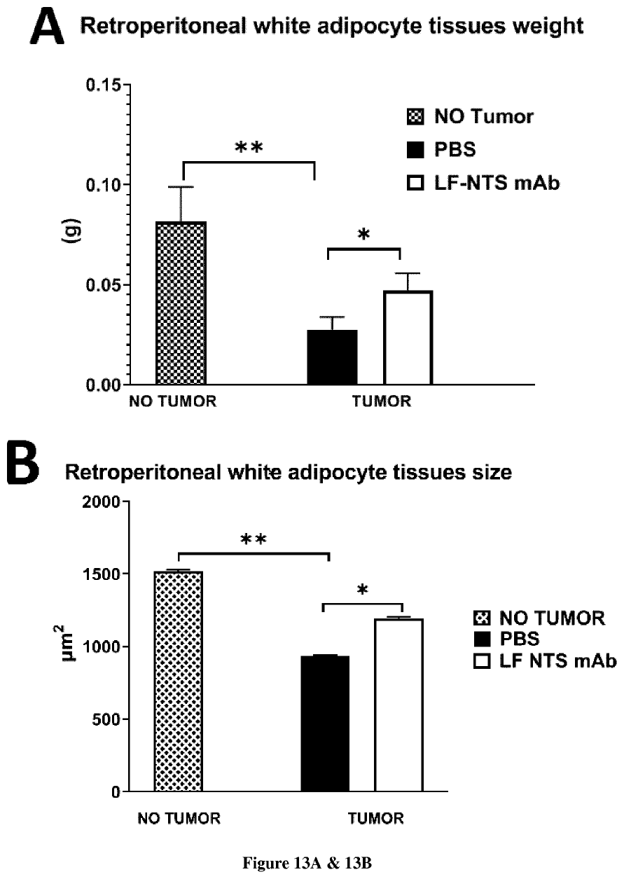

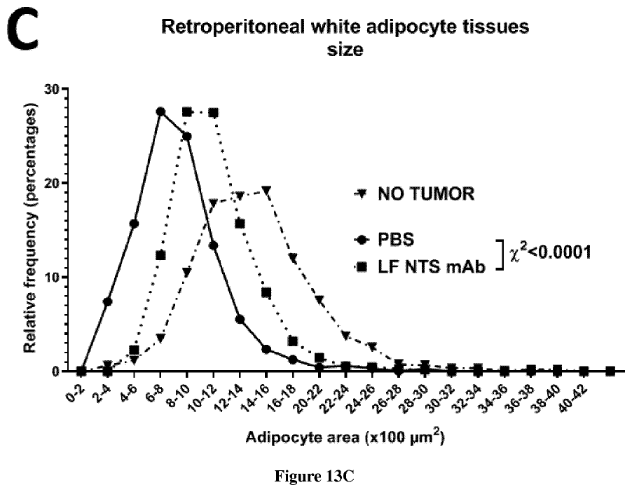

Epididymal (EWAT) and the retro-peritoneal (RPWAT) white adipose tissues were weighted after sacrifice. In animals bearing tumors, both EWAT and RPWAT were lighter as compared to animals nonbearing tumors (FIGS. 12A & 13A), indicating that this model is able to induce cachexia. When animals are treated with LF-NTS mAb the EWAT and RPWAT were heavier (FIGS. 12A & 13A). We calculated the size of the adipocytes of EWAT and RPWAT. We observed that the adipocytes from animals bearing tumors are small with the highest frequency at 600 to 800 μm², and 400 to 600 μm² for EWAT and RPWAT, respectively (FIGS. 12B and 13C & 13B and 13C). Whereas, in animals bearing tumors, treated with LF NTS mAb, the size of EWAT was equivalent to the EWAT from nonbearing tumors animals, with a highest frequency of 1300 μm² (FIGS. 12B and 12C). LF-NTS mAb prevent partially the emptying of the adipocytes of the RPWAT. The highest frequency was around 900 μm² and 1500 μm² for animals treated with LF NTS mAb and animals without tumors, respectively (FIGS. 13B and 13C). These observations on adipocytes confirm the hypothesis as LF NTS mAb counteract cachexia induced by cancer.

### REFERENCES

Throughout this application, various references describe the state of the art to which this invention pertains. The disclosures of these references are hereby incorporated by reference into the present disclosure.

1. Evans W J, Morley J E, Argiles J, Bales C, Baracos V, Guttridge D, Jatoi A, Kalantar-Zadeh K, Lochs H, Mantovani G, Marks D, Mitch W E, Muscaritoli M, Najand A, Ponikowski P, Rossi Fanelli F, Schambelan M, Schols A, Schuster M, Thomas D, Wolfe R, Anker S D. Cachexia: a new definition. Clinical nutrition (Edinburgh, Scotland) 2008; 27: 793-799.

2. Argiles J M, Anker S D, Evans W J, Morley J E, Fearon K C, Strasser F, Muscaritoli M, Baracos V E. Consensus on cachexia definitions. Journal of the American Medical Directors Association 2010; 11: 229-230.

3. Carraway R, Leeman S E. Characterization of radioimmunoassayable neurotensin in the rat. Its differential distribution in the central nervous system, small intestine, and stomach. The Journal of biological chemistry 1976; 251: 7045-7052.

4. Polak J M, Sullivan S N, Bloom S R, Buchan A M, Facer P, Brown M R, Pearse A G. Specific localisation of neurotensin to the N cell in human intestine by radioimmunoassay and immunocytochemistry. Nature 1977; 270: 183-184.

5. Kitabgi P, Freychet P. Effects of neurotensin on isolated intestinal smooth muscles. European journal of pharmacology 1978; 50: 349-357.

6. Blackburn A M, Bloom S R. A radioimmunoassay for neurotensin in human plasma. The Journal of endocrinology 1979; 83: 175-181.

7. Kitabgi P, Checler F, Mazella J, Vincent J P. Pharmacology and biochemistry of neurotensin receptors. Reviews in clinical & basic pharmacology 1985; 5: 397-486.

8. Vincent J P. Neurotensin receptors: binding properties, transduction pathways, and structure. Cellular and molecular neurobiology 1995; 15: 501-512.

9. Schroeder L E, Leinninger G M. Role of central neurotensin in regulating feeding: Implications for the development and treatment of body weight disorders. Pharmaceutics 2018; 1864: 900-916.

10. Christ-Crain M, Stoeckli R, Ernst A, Morgenthaler N G, Bilz S, Korbonits M, Struck J, Bergmann A, Muller B, Keller U. Effect of gastric bypass and gastric banding on proneurotensin levels in morbidly obese patients. The Journal of clinical endocrinology and metabolism 2006; 91: 3544-3547.

11. Ratner C, Skov L J, Raida Z, Bachler T, Bellmann-Sickert K, Le Foll C, Sivertsen B, Dalboge L S, Hartmann B, Beck-Sickinger A G, Madsen A N, Jelsing J, Holst J J, Lutz T A, Andrews Z B, Holst B. Effects of Peripheral Neurotensin on Appetite Regulation and Its Role in Gastric Bypass Surgery. The Journal of neuroscience: the official journal of the Society for Neuroscience 2016; 157: 3482-3492.

12. Barchetta I, Ciccarelli G, Cimini F A, Ceccarelli V, Orho-Melander M, Melander O, Cavallo M G. Association between systemic leptin and neurotensin concentration in adult individuals with and without type 2 diabetes mellitus. 2018.

13. Rostene W H, Alexander M J. Neurotensin and neuroendocrine regulation. Frontiers in neuroendocrinology 1997; 18: 115-173.

14. Mazzocchi G, Malendowicz L K, Rebuffat P, Gottardo G, Nussdorfer G G. Neurotensin stimulates CRH and ACTH release by rat adrenal medulla in vitro. Neuropeptides 1997; 31: 8-11.

15. Malendowicz L K, Nussdorfer G G, Markowska A, Tortorella C, Nowak M. Investigations on the acute effects of neuropeptides on the pituitary-adrenocortical function in normal and cold-stressed rats. II. Neurotensin and neuromedin N. Experimental and toxicologic pathology: official journal of the Gesellschaft fur Toxikologische Pathologie 1995; 47: 41-45.

16. Ward E S, Gussow D, Griffiths A D, Jones P T, Winter G. Binding activities of a repertoire of single immunoglobulin variable domains secreted from Escherichia coli. Nature 1989; 341: 544-546.

17. Holt L J, Herring C, Jespers L S, Woolven B P, Tomlinson I M. Domain antibodies: proteins for therapy. Trends in biotechnology 2003; 21: 484-490.

1. A method of preventing weight loss, muscle loss and/or protein blood level decrease in a subject in need thereof comprising administering to the subject a therapeutically effective amount of an inhibitor of NTSR1 activation or expression.

2. The method of claim 1 wherein the subject is underweight.

3. The method of claim 1 wherein the subject suffers from a wasting disorder selected from the group consisting of anorexia cachexia, anorexia of the aged, anorexia nervosa, cachexia associated with cancer, cachexia associated with AIDS, cachexia associated with heart failure, cachexia associated with cystic fibrosis, cachexia associated with rheumatoid arthritis, cachexia associated with kidney disease, cachexia associated with chronic obstructive pulmonary disease (COPD), cachexia associated with ALS, cachexia associated with renal failure or cachexia associated, and other disorders associated with aberrant appetite, fat mass, energy balance, and/or involuntary weight loss.

4. The method of claim 1 wherein the subject suffers from cachexia.

5. The method of claim 4 wherein the subject suffers from cancer.

6. The method of claim 1 wherein the inhibitor of NTSR1 activation or expression is an antibody.

7. The method of claim 6 wherein the antibody is humanized or human antibody.

8. The method of claim 6 wherein the antibody contains the heavy chain CDRs of the heavy chain variable region of NTSp27.7.4 (SEQ ID NO:3) as represented by SEQ ID NO:7-9 or FLp26-8.2 (SEQ ID NO:4), as represented by SEQ ID NO:13-15.

9. The method of claim 6 wherein the antibody of the present invention comprises the light chain CDRs of the light chain variable region of NTSp27.7.4 (SEQ ID NO:5) as represented by SEQ ID NO:10-12 or FLp26-8.2 (SEQ ID NO:6) as represented by SEQ ID NO:16-18.

10. The method of claim 6 wherein the antibody comprises the heavy chain CDRs of the heavy chain variable region of NTSp27.7.4 (SEQ ID NO:3) and the light chain CDRs of the light chain variable region of NTSp27.7.4 (SEQ ID NO:4).

11. The method of claim 6 wherein the antibody comprises the heavy chain CDRs of the heavy chain variable region of FLp26-8.2 (SEQ ID NO:5) and the light chain CDRs of the light chain variable region of FLp26-8.2 (SEQ ID NO:6).

12. The method of claim 8 wherein the antibody binds to an epitope comprising 1, 2, 3, 4, 5, 6, 7, 8, 9, 10, 11, 12, 13, 14, 15 amino acid residues from amino acid residues 123 to 137 of SEQ ID NO: 2 or of SEQ ID NO: 20.

13. The method of claim 1 wherein a competing antibody cross-competes for binding to the epitope comprising amino acid residues from amino acid residues 123 to 137 of SEQ ID NO: 2 or of SEQ ID NO: 20 with the monoclonal antibody comprising a heavy chain comprising the following CDRs: i) the H-CDR1 of NTSp27.7.4 as set forth in SEQ ID NO: 7, ii) the H-CDR2 of NTSp27.7.4 as set forth in SEQ ID NO: 8 and iii) the H-CDR3 of NTSp27.7.4 as set forth in SEQ ID NO: 9 and a light chain comprising the following CDRs: i) the L-CDR1 of NTSp27.7.4 as set forth in SEQ ID NO 10, ii) the L-CDR2 of NTSp27.7.4 as set forth in SEQ ID NO: 11 and iii) the L-CDR3 of NTSp27.7.4 as set forth in SEQ ID NO: 12.

14. The method of claim 1 wherein a competing antibody cross-competes for binding to the epitope comprising amino acid residues from amino acid residues 123 to 137 of SEQ ID NO: 2 or of SEQ ID NO: 20 with the monoclonal antibody comprising a heavy chain comprising the following CDRs: i) the H-CDR1 FLp26-8.2 as set forth in SEQ ID NO: 13, ii) the H-CDR2 FLp26-8.2 as set forth in SEQ ID NO: 14 and iii) the H-CDR3 FLp26-8.2 as set forth in SEQ ID NO:15 and light chain comprising the following CDRs: i) the L-CDR1 FLp26-8.2 as set forth in SEQ ID NO:16, ii) the L-CDR2 FLp26-8.2 as set forth in SEQ ID NO:17 and iii) the L-CDR3 FLp26-8.2 as set forth in SEQ ID NO:18.


SEQUENCE LISTING (20 sequences)

SEQ ID NO: 1 - protein, 13 aa, Homo sapiens
QLYENKPRRPYIL

SEQ ID NO: 2 - protein, 170 aa, Homo sapiens
MMAGMKIQLVCMLLLAFSSWSLCSDSEEEMKALEADFLTNMHTSKISKAHVPSWKMTLLNVCSLVNNLNSPAEETGEVHEEELVARRKLPTALDGFSLEAMLTIYQLHKICHSRAFQHWELIQEDILDTGNDKNGKEEVIKRKIPYILKRQLYENKPRRPYILKRDSYYY

SEQ ID NO: 3 - protein, 122 aa, Mus musculus
DVKLVESGGGLVKLGGSLKLSCAASGFTFSGYYMSWVRQTPEKRLELVAAINNYGDNTNYPDTVKGRFSVSRDNAKNTLYLEMNSLKSEDTALYYCARLANYANQRGAMDYWGQGTSVTVSS

SEQ ID NO: 4 - protein, 107 aa, Mus musculus
DIQMTHTTSSLSASLGDRVTISCRASQDIANYLNWYQQKPDGTVTLLIYYTSRLHSGVPSRFSGSGSGTDYSLTINNLDQEDIATYFCQQGYTLPPTFGGGTKLEIK

SEQ ID NO: 5 - protein, 116 aa, Mus musculus
QIQLVQSGPELKKPGETVKISCKASGYTFTNYGMNWVKQAPGKGLKWMGWITTNTGEPTYAEEFKGRFAFSLETSASTAYLQINNLKNEDTATYFCARRAFAMDYWGQGTSVTVSS

SEQ ID NO: 6 - protein, 112 aa, Mus musculus
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPYTFGGGTKLEIK

SEQ ID NO: 7 - protein, 10 aa, Mus musculus
GFTFSGYYMS

SEQ ID NO: 8 - protein, 17 aa, Mus musculus
AINNYGDNTNYPDTVKG

SEQ ID NO: 9 - protein, 13 aa, Mus musculus
LANYANQRGAMDY

SEQ ID NO: 10 - protein, 11 aa, Mus musculus
RASQDIANYLN

SEQ ID NO: 11 - protein, 7 aa, Mus musculus
YTSRLHS

SEQ ID NO: 12 - protein, 9 aa, Mus musculus
QQGYTLPPT

SEQ ID NO: 13 - protein, 10 aa, Mus musculus
GYTFTNYGMN

SEQ ID NO: 14 - protein, 17 aa, Mus musculus
WITTNTGEPTYAEEFKG

SEQ ID NO: 15 - protein, 7 aa, Mus musculus
RAFAMDY

SEQ ID NO: 16 - protein, 16 aa, Mus musculus
RSSKSLLHSNGNTYLY

SEQ ID NO: 17 - protein, 7 aa, Mus musculus
RMSNLAS

SEQ ID NO: 18 - protein, 9 aa, Mus musculus
MQHLEYPYT

SEQ ID NO: 19 - protein, 6 aa, Homo sapiens
KIPYIL

SEQ ID NO: 20 - protein, 150 aa, Homo sapiens
MMAGMKIQLVCMLLLAFSSWSLCSDSEEEMKALEADFLTNMHTSKISKAHVPSWKMTLLNVCSLVNNLNSPAEETGEVHEEELVARRKLPTALDGFSLEAMLTIYQLHKICHSRAFQHWELIQEDILDTGNDKNGKEEVIKRKIPYILKR